(19) United States
(12) Patent Application Publication
(10) Pub. No.: US 2005/0273062 A1
(43) Pub. Date: Dec. 8, 2005

(54) Infusion set

(76) Inventors: Olof Franksson, Stockholm (SE); Johan Rinman, Aneby (SE); Robert Axelsson, Granna (SE)

Correspondence Address: WINSTON & STRAWN LLP, 1700 K STREET, N.W., WASHINGTON, DC, 20006, US

(21) Appl. No.: 11/181,413

(22) Filed: Jul. 13, 2005

(30) Foreign Application Priority Data
Jan. 20, 2003 (SE) SE 0300137-7
Jan. 20, 2004 (WO) PCT/EP04/00417

(51) Int. Cl. A61M 5/00

(52) U.S. Cl. 604/254

(57) ABSTRACT

An infusion set for administering an infusion fluid to a patient. The infusion set includes a fluid container for the infusion fluid, an injection needle for injecting the infusion fluid into the patient's body, a flow regulator having a housing with an inlet, which communicates with the fluid container, and an outlet, which communicates with the injection needle via a fluid duct, at least one fluid chamber in the housing, at least one floater placed in the at least one fluid chamber. Moreover the infusion set comprises an inlet throttle and/or closure valve that includes a valve seat formed on the housing at the inlet and a corresponding inlet valve body connected with the floater at an axial distance from the at least one floater by at least one connector, an air gap formed between the inlet valve body and the floater, and at least one opening in the at least one connector for allowing fluid communication between the inlet throttle and/or closure valve and the housing. The flow regulator automatically regulates, with optimal comfort and safety for the patient, the flow of fluid by means of a simple and inexpensive construction allowing the nurses to watch and evaluate the progress of the infusion process through the window.

### CROSS-REFERENCE TO RELATED APPLICATIONS

This application is a continuation of International Application PCT/EP2004/000417 filed Jan. 20, 2004, the entire content of which is expressly incorporated herein by reference thereto.

### BACKGROUND

The present invention relates to an infusion set for administering an infusion fluid to a patient. The infusion set comprises a fluid container for the infusion fluid, an injection needle for injecting the infusion fluid into the patient's body, a flow regulator having a housing with an inlet, which communicates with the fluid container, and an outlet, which communicates with the injection needle via a fluid duct, at least one fluid chamber in the housing, and at least one floater placed in the at least one fluid chamber.

Infusion sets are widely used for the treatment of patients who need infusion fluids like e.g. medicine, plasma or blood. The infusion fluid is supplied to the patient by means of the injection needle, which is introduced into e.g. a vein of the patient while the fluid container is hanging on e.g. a stand on a suitable level over the injection needle.

The weight of the column of fluid in the fluid duct provides the necessary force for driving the infusion fluid through the duct from the fluid container to the injection needle and via this into the body of the patient while overcoming the resistance, which especially the body of the patient offers against the flow of the infusion fluid.

The flow resistance will frequently increase as the infusion process proceeds. When using a conventional infusion set the resistance in the fluid duct is therefore manually regulated concurrently with the changing of the flow resistance in the body of the patient by means of a tube clamp.

Such conventional infusion sets usually also have a transparent drip chamber indicating the progress of the infusion process. By, from time to time, visually observing the frequency of drops entering the drip chamber, it is possible for the nurses to intervene in the process by manipulating the tube clamp in accordance with the observations made.

The infusion process can, without these manually performed adjustments, easily take an unfavourable course, which consequently could injure the patient being supplied too much or too little infusion fluid per unit of time.

It is especially important that the process is stopped in proper time to prevent the patient's blood from flowing back into the infusion set when the fluid container, e.g. a plastic bag, is running out of infusion fluid.

These frequent manual adjustments are inconvenient for the patient and are moreover not always carried out with the required care and accuracy by the often very busy nurses.

The manual adjustment of the infusion process is furthermore a difficult, tiresome and time-consuming work taking up costly time for the nurses.

Over the years attempts have therefore been made to develop a more or less automatically functioning flow regulator for an infusion set.

From the patent JP 61247469 is known a flow regulator for a fluid and blood infusion set. This flow regulator has a floater engaging a valve seat for automatically closing the outlet of the chamber when the fluid container is empty or nearly empty, thereby preventing the patient's blood from flowing back into the infusion set. This regulator is not able to automatically regulating the flow.

Another flow regulator is known from U.S. Pat. No. 6,337,631 B1. The position of the floater in the housing of this regulator is monitored by means of a detecting unit, which is electrically connected to an alarm unit. The floater sinks in the housing when the infusion fluid in the fluid container is running out, whereby the alarm unit is activated by the detecting unit. The alarm calls a nurse for stopping the process. This regulator serves only for stopping the infusion process but is not able to automatically regulating the flow of fluid during the infusion process. The regulator is furthermore complicated and costly.

From U.S. Pat. No. 4,096,879 is known an apparatus having two regulators, which in co-operation are able to regulate the flow of fluid to the patient. This apparatus is very complicated and costly and is moreover difficult to operate.

U.S. Pat. No. 3,667,464 discloses an infusion set where a floater with a valve part is provided in the upper part of the infusion set and a section for monitoring the drops is provided in the lower part of the infusion set. However, the invention as described herein does not prevent the blood from the patient from flowing back into the infusion set. In addition, a separate section is required for monitoring the drop rate, which will make the infusion set relatively large and complex. Thus, improved devices are desired.

### SUMMARY OF THE INVENTION

In one aspect according to the invention is provided an infusion set of the type mentioned in the opening paragraph, which has an automatically functioning flow regulator.

In a second aspect according to the invention is provided an infusion set of the type mentioned in the opening paragraph arranged in such a way that the nurses visually can watch the infusion process.

In a third aspect according to the invention is provided an infusion set of the type named in the opening paragraph, which is adapted to function with optimal comfort and safety for the patient.

In a fourth aspect according to the invention is provided an infusion set of the type mentioned in the opening paragraph, which has a simple and inexpensive construction.

These aspects are achieved in a flow regulator according to the invention that comprises an inlet throttle and/or closure valve consisting of a valve seat formed on the housing at the inlet and a corresponding valve body connected with the floater at an axial distance from the at least one floater by means of at least one connector, an open air gap formed between the valve body and the floater, an at least one opening in the at least one connector for allowing fluid communication between the inlet throttle and/or closure valve and the housing.

When the housing is transparent it is possible to follow the progress of the infusion process by visually watching drops of fluid falling in the housing.

The flow regulation is carried out in response to the flow resistance in especially the body of the patient. The valve opens when the flow resistance is relatively small and throttles or stops the fluid flow from the fluid container to the housing when the flow resistance grows such that the level of fluid in the housing rises and the floater is thereby brought closer to the inlet.

The maximum allowable stroke of the floater in the housing is preferably relatively short thereby avoiding unwanted time lag in the regulation of the fluid flow. If, on the contrary, the stroke is relatively long it will take a relatively long time to obtain a relatively small change in the fluid flow, since a long stroke requires a relatively large amount of fluid to be filled into the housing and the fluid flow to the body of the patient normally is relatively slow, whereby the fluid flow can pulsate in a way, which could be very unpleasant for the patient to experience. If occasion should arise the patient could also get injured. The comfort of the patient requires the fluid to be supplied gently and steadily.

The allowed maximal stroke in the housing of the regulator could advantageously be between 0.1 and 15 mm, preferably between 0.5 and 5 mm, and especially between 1.5 and 2 mm.

A relatively short distance between the valve body and the valve seat in all positions of the floater in the housing will, owing to the surface tension of the fluid, cause the fluid, which leaves the inlet, to bridge over the variable gap between the mouth of the inlet and the top of the inlet valve body instead of falling as drops into the housing as in a conventional drip chamber.

The fluid in the fluid bridge will successively overflow the top of the inlet valve body and, in the form of a thin layer, follow the surface of this to the bottom due to the adherence between the fluid and the surface of the inlet valve body.

The surface tension of the fluid and the adherence between the fluid and the inlet valve body allows the fluid, which has reached the bottom of the inlet valve body, to accumulate to a certain size, whereupon the accumulated fluid will leave the inlet valve body in the form of a drop, which is allowed to develop when the distance between the inlet valve body and the floater is larger than the size of a drop.

The quantity of fluid, which is successively supplied to the inlet valve body, is dependent on the height of the fluid bridge, which changes concurrently with the movements of the floater in the housing. The above-mentioned construction, which allows the fluid bridge to come into existence, is therefore an important part of the automatic regulation effect of the flow regulator according to the invention.

The inlet valve body is, in an advantageous embodiment according to the invention, formed with a conical surface having a tip facing the floater, thereby ensuring that well-defined and uniform drops are formed at the middle of the housing allowing the nurses to safely observe the drops and evaluate the progress of the infusion process when the housing is transparent.

In a preferred embodiment a cavity can be formed in the inlet valve body for catching and equalizing the supply of fluid to the tip of the conical surface of the inlet valve body by overflowing a free edge of the wall of the cavity or flowing through openings in this wall.

When the connector has a slanting part extending outwards from the mainly conical surface of the inlet valve body and slanting against the inlet of the housing it is advantageously obtained that the part of the automatic regulation functioning is not disturbed because the fluid or some of the fluid can run down along the connector instead of falling as drops from the valve body as is desired.

For centring the floater and thereby the inlet valve body in the housing, a guide pin, which is formed on the top of the valve body, can extend into the inlet of the housing.

The flow regulator according to the invention can in addition to the above mentioned inlet throttle and/or closure valve also comprise an outlet throttle and/or closure valve consisting of an outlet valve seat formed on the housing at the outlet and a corresponding outlet valve body formed on the floater, thereby improving the functioning of the automatic regulation of the flow regulator.

This outlet throttle and/or closure valve will close the outlet of the housing when the fluid container is discharged or nearly discharged, whereby blood from the patient is prevented from flowing backwards into the infusion set, because the column of fluid in the fluid duct and injection needle will be maintained and thereby sustain the necessary overpressure in relation to the blood pressure in e.g. a vein of the patient.

The flow regulator can, for further improving the regulation accuracy, comprise an elastic means such as an open prestressed spring, which functions between the valve body of the outlet throttle and/or closure valve and the housing for urging the outlet valve body against the outlet valve seat, thereby securing that the outlet valve first opens when the fluid in the housing has obtained a predetermined level.

When a ventilation opening is formed in the inlet end of the housing there will always be the same pressure inside the housing of the flow regulator as outside. The flow resistance in e.g. a vein of the patient does therefore not directly influence the flow of fluid from the fluid container to the flow regulator.

The infusion set can in a simple embodiment comprise a housing with only one fluid chamber and only one floater placed in this chamber.

The flow regulator can however, in an advantageously other embodiment, comprise a partition dividing the housing into a first housing part with the inlet, and defining a first fluid chamber, and a second housing part with the outlet, and defining a second fluid chamber, an outlet control valve arranged in the partition for allowing fluid communication between the two chambers, and a first—and second floater placed in the first and second fluid chamber, respectively. By means of this construction is added further possibilities for being able precisely to regulate the fluid flow from the fluid container to the patient's body.

The outlet control valve for allowing fluid communication between the two chambers can advantageously comprise at least one first flow passage eccentrically formed in the bottom of the first housing part and at least one second flow passage, which in cooperating with the first flow passage is formed in the partition, which furthermore is rotatably connected with the at least first housing part, whereby a very accurate regulation of the fluid flow through the flow regulator can be obtained by simply rotating the partition relatively to the first housing part.

The partition can in an advantageous embodiment comprise a tubular part rotatable connected with at least the first housing part and a wall extending across the inner side of the tubular part, whereby the at least second flow passage is formed in this wall.

A second ventilation opening can be formed in the tubular part of the partition or in the upper part of the second housing part for allowing communication between the second housing part and the surroundings such that there also will be the same pressure inside the second housing part as outside the housing part, whereby the flow resistance in e.g. a vein of the patient does not directly influence the flow of fluid from the fluid container to the flow regulator.

A filter can furthermore be inserted into one or both of the ventilation openings of the housing for preventing contaminations from the environment to access any of the housings and thereby contaminate the infusion fluid.

### BRIEF DESCRIPTION OF THE DRAWINGS

The invention will be explained in greater details below where further advantageous properties and example embodiments are described with reference to the drawings, in which

FIG. 1 shows in perspective a first embodiment for an infusion set according to the invention,

FIG. 2 shows in perspective on a larger scale, partly in section a flow regulator for the infusion set shown in FIG. 1,

FIG. 3 shows in section on a still larger scale a detail of the flow regulator shown in FIG. 2 with an inlet valve of the flow regulator,

FIG. 4 shows in perspective another embodiment for an infusion set according to the invention,

FIG. 5 shows in section on a larger scale a flow regulator for the infusion set shown in FIG. 4,

FIG. 6 shows in section on a still larger scale a detailed view with a an inlet valve of the flow regulator shown in FIG. 5,

FIG. 7 shows in section on the same scale another a detailed view with an outlet valve of the flow regulator shown in FIG. 5 in closed position,

FIG. 8 shows the same with the outlet valve in open position,

FIG. 9 shows in section on the same scale a third detail with a control valve of the flow regulator shown in FIG. 5, and

FIG. 10 shows a section of the flow regulator taken along the line X-X in FIG. 9.

### DETAILED DESCRIPTION OF THE PREFERRED EMBODIMENTS

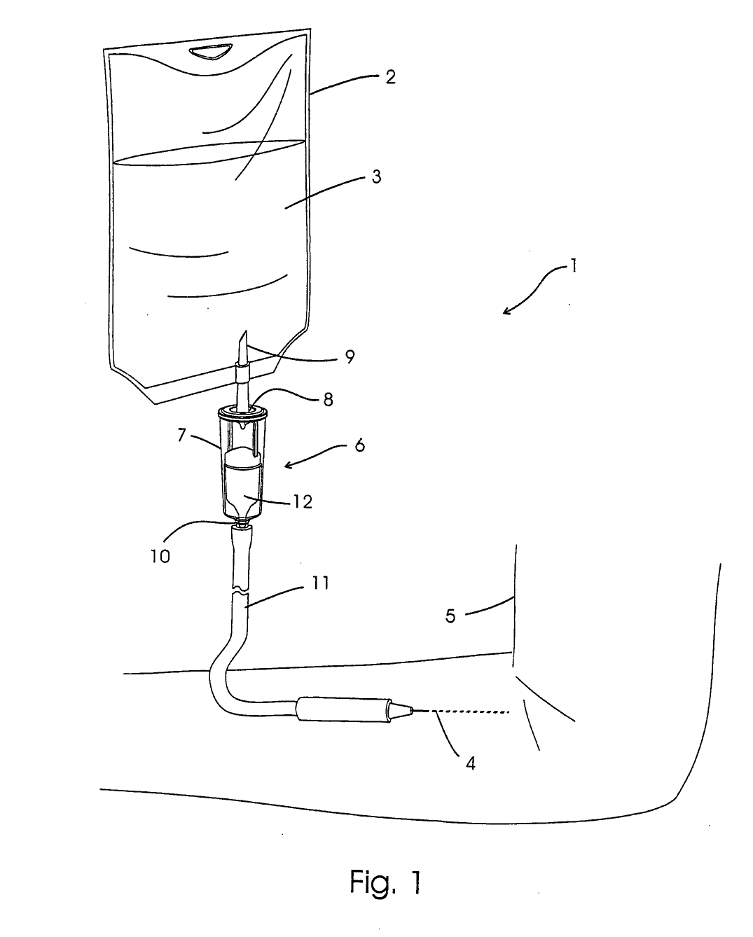

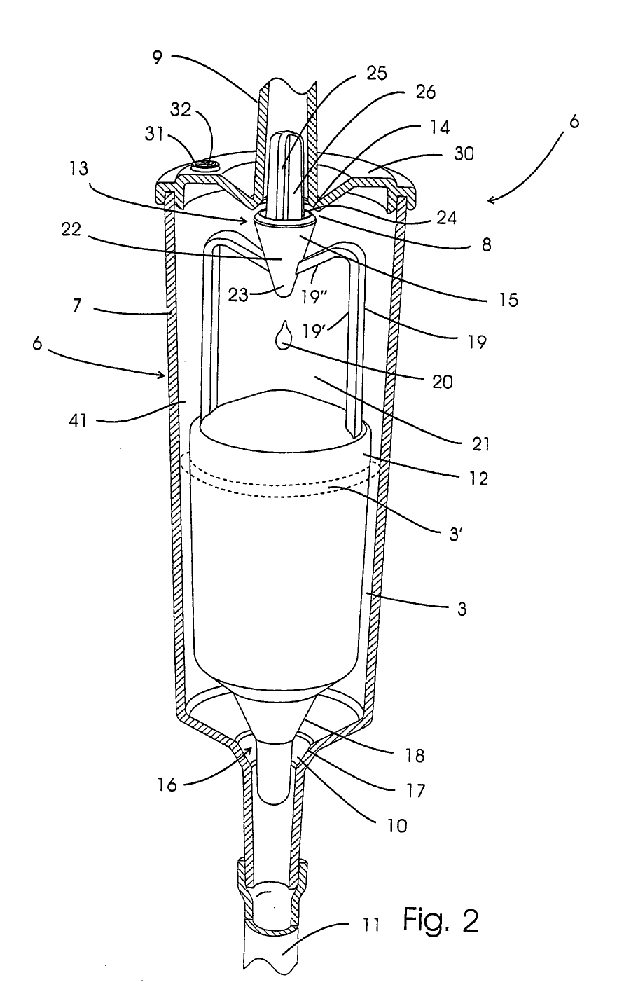

FIGS. 1-3 shows a first embodiment for an infusion set 1 according to the invention comprising a fluid container 2 for the infusion fluid 3, an injection needle 4 for injecting the infusion fluid into the patient's body 5, a flow regulator 6 having in this case a transparent housing 7 with an inlet 8, which communicates with the fluid container 2 via a spike 9, and an outlet 10, which communicates with the injection needle 4 via a fluid duct 11.

The fluid container 2, which is typically a plastic bag, is hung up on a support (not shown) on a sufficiently high level above the patient's body 5 for obtaining the overpressure needed for driving the fluid into e.g. a vein of the patient. This overpressure is provided by the weight of the column of fluid in the infusion set being in this position.

In the housing 7 is placed a floater 12, an inlet throttle and/or closure valve 13 consisting of an inlet valve seat 14 formed on the housing 7 at the inlet 8 and a corresponding inlet valve body 15, and in this case also an outlet throttle and/or closure valve 16 consisting of an outlet valve seat 17 formed on the housing 7 at the outlet 10 and a corresponding outlet valve body 18.

The inlet valve body 15 is connected with the floater 12 by means of two legs 19, which extend at a distance from each other along the housing. Each leg 19 has a first part 19′ connected with the floater and a second part 19″ connected with the inlet valve body 15. The two parts 19′ and 19″ forms an acute angle α with each other in such a way that the second part 19″ declines towards the outlet of the housing at the connection of the legs 19 with the inlet valve body 15.

The legs 19 also have such a length that the distance between the inlet valve body 15 and the floater 12 becomes larger than the size of a fluid drop 20. The legs 19 define a window 21 enabling the nurses to see such drops 20 when the housing 7 is transparent.

The outlet valve body 18 is formed directly on the floater 12 and has a conical form, while the outlet valve seat 17 has a corresponding conical form.

The inlet valve body 15 has a conical surface 22 terminating in a tip 23, which is facing the floater 12. A cavity 24 is formed in the topmost part of the inlet valve body 15 opposite the tip 23.

A guide pin 25 for centring the inlet valve body 15 in relation to the inlet valve seat 14 extends into the inlet 8 of the housing 7 from the bottom of the cavity 24. Longitudinal grooves 26 for allowing the fluid from the fluid container to freely enter the cavity 24 is formed in the guide pin 25.

At the end of the infusion process there is no fluid or at least very little fluid left in the fluid container. The column of fluid in the infusion set will then sink, whereby the overpressure in relation to the pressure in the body of the patient gradually decreases until it is smaller than the pressure. At this stage the flow direction of the fluid will be reversed resulting in e.g. blood from the body flowing undesirably into the infusion set.

This adverse situation is, however, avoided by using the flow regulator 6 according to the invention, since the outlet valve body 18 of this regulator 6 closes the corresponding outlet valve seat 17 as soon as the level of the fluid in the housing 7 of the regulator 6 is so low that the remaining buoyancy is too little to bear up the floater 12. The column of fluid in the fluid duct 11 therefore remains standing thereby advantageously securing that the necessary overpressure exists as long as the injection needle 4 is present in the body 5 of the patient.

During the infusion process the inlet and outlet valves 13 and 16 are open at the same time or are alternating between open and closed in order to regulate the flow of fluid 3 to the body 5 of the patient.

The housing 7 of the regulator 6 will be emptied of fluid 3 if the fluid 3 flows too fast into the body 5 of the patient resulting in the outlet valve 16 temporary stopping the fluid flow and thereby regulates the fluid flow.

Another situation occurs when the body 5 of the patient is not able to fully receive the supplied fluid whereupon the level of fluid in the housing 7 of the regulator 6 rises and the inlet valve body 15 is brought closer to the valve seat 14 or is abutting it.

The regulation of the fluid flow can more or less take place by only throttling the fluid flow depending on the fluctuating distance between the valve bodies 15,18 and their corresponding valve seats 14,17 and/or by alternating between opening and closing the valves 13,16.

The maximum allowable stroke of the floater 12 in the housing 7 is relatively short to avoid an unpleasant and possibly injurious time lag in the regulation process because the housing 7 of the regulator 6 needs to be filled with too much fluid for bringing the floater 12 from one regulation position to another when the stroke on the contrary is relatively long.

The stroke is therefore chosen to have a size such that the valves in just able to open sufficiently to allow the maximal wanted fluid flow through the housing of the regulator.

The floater 12 can in agreement with this choice in a preferred embodiment have a maximal stroke in the housing of the regulator between 0.1 and 15.0 mm, preferably between 0.5 and 5 mm, and especially between 1.5 and 2.0 mm, whereby the top of the inlet valve body 15 will be relatively close to its inlet valve seat 14 in all regulation positions of the floater 12.

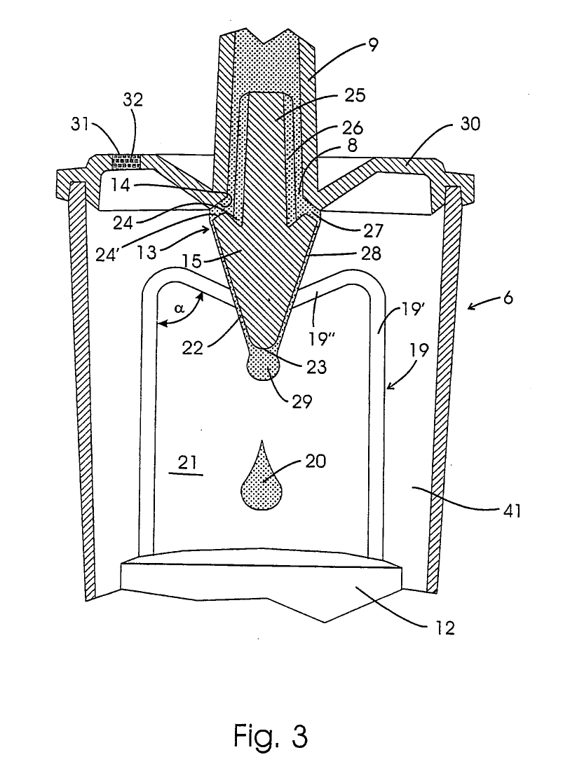

As best seen in FIG. 3, the fluid will, owing to the surface tension, form a bridge 27 between the inlet valve seat 14 and the top of the outlet valve body 15, whereby the cavity is also filled with a reservoir of fluid.

The fluid will via the upper edge 24′ overflow the top of the inlet valve body 15 and due to the adherence between the fluid and the surface of the inlet valve body 15 run in the form of a thin layer 28 down along the conical surface 22 of the inlet valve body 15 to its tip 23.

The fluid 29 at the tip 23 accumulates to a certain size depending on the surface tension of the fluid 3, the adherence between the fluid 3 and the surface of the inlet valve body 15, and the shape of the tip 23.

When the accumulated fluid has achieved this certain size the fluid is by means of the gravity released from the tip 23 in the form of a drop 20 falling down against the floater 12.

Due to the window 21 defined by the legs 19, the nurses can advantageously monitor and evaluate the progress of the infusion process by visually observing the frequency of the falling drops 20 when the housing is transparent.

The height of the fluid bridge 27 changes concurrently with the movements of the floater 12 in the housing 7, thereby supplying the inlet valve body 15 with correspondingly changing quantities of fluid.

This effect implies that the regulation of the fluid flow is carried out with a high grade of accuracy, which is beneficial for the comfort and safety of the patient.

In the cap 30 on top of the fluid container 2 a ventilation opening 31 is formed. The opening 31 is equipped with a filter 32 adapted to prevent contaminants from the environment from penetrating into the regulator 6 and thereby into the infusion fluid.

The pressure in the housing 6 is therefore always the same as in the environment, whereby the flow resistance in e.g. a vein of the patient does not directly influence the flow of fluid from the liquid container to the flow regulator 6.

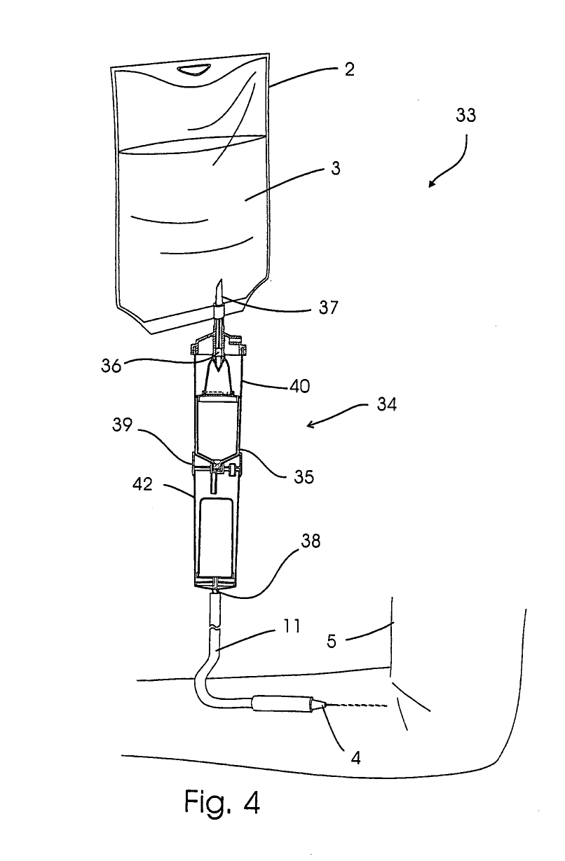

FIGS. 4-10 shows another embodiment for an infusion set 33 according to the invention comprising a fluid container 2 for the infusion fluid 3, an injection needle 4 for injecting the infusion fluid 3 into the patient's body 5, a flow regulator 34 having in this case a transparent housing 35 with an inlet 36, which communicates with the fluid container 2 via a spike 37, and an outlet 38, which communicates with the injection needle 4 via a fluid duct 11.

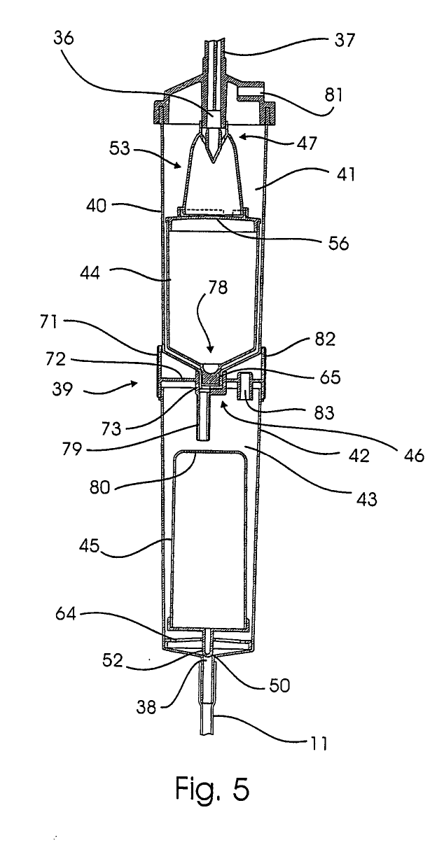

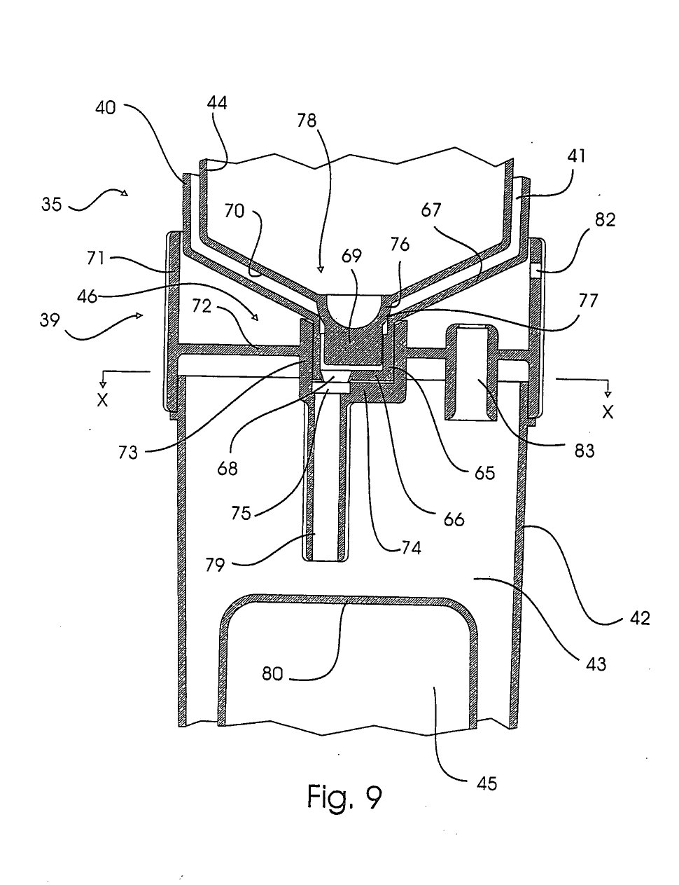

In this embodiment a partition 39 divides the housing 35 of the flow regulator 34 into a first housing part 40, which has an inlet 36 and is defining a first fluid chamber 41, and a second housing part 42, which has an outlet 38 and is defining a second fluid chamber 43. A first floater 44 is placed in the first fluid chamber 41, while a second floater 45 is placed in the second fluid chamber 43. As seen in FIGS. 5 and 9 the two chambers 41,42 are in communication via a control valve 46.

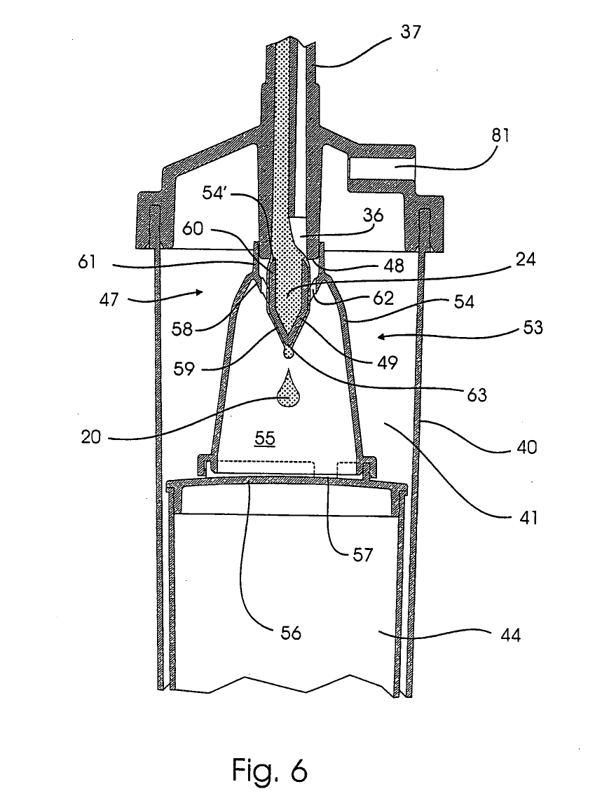

As seen best in FIGS. 5 and 6 an inlet throttle and/or closure valve 47 is arranged at the inlet 36. The inlet throttle and/or closure valve 47 consists of an inlet valve seat 48 formed on the first housing part 40 and a corresponding inlet valve body 49 formed on the first floater 44.

As seen in FIGS. 5, 7 and 8 an outlet throttle and/or closure valve 50 is arranged at the outlet 38, the outlet throttle and/or closure valve 50 consists of an outlet valve seat 51 formed on the second housing part 42 and a corresponding outlet valve body 52 on the second floater 45.

As best seen in FIG. 6 the inlet valve body 49 of the inlet valve 47 is connected with the first floater 44 via a connector 53 at an axial distance from the floater 44. The connector 53 has a wall 54, which at least partly is enclosing a cavity 55. In the wall 54 is, preferably close to the top 56 of the first floater 14, formed a number of through openings 57 for allowing the cavity 55 to communicate with the fluid chamber 41 at the other side of the wall 54.

The wall 54 of the connector 53 has an upper part 58 extending in a slanting way downwards to the inlet valve body 49, which has a lower conical drip part 59 and an upper closing part 60 for closing the inlet valve 47 when engaging the valve seat 48.

The connector 53 has an annular projection 61 extending against or past the valve seat 48 of the first housing part 40. A number of through openings 62 is formed in the slanting upper part 58 of the wall 54 of the connector 53 in an area between the projection 61 and the upper closing part 60 of the inlet valve body 52.

When the inlet valve 47 is open and the infusion set 33 is in use the infusion fluid fills the cavity 24 and overflows the free edge 54′ of the upper slanting wall part 58 and runs out via the through openings 62 to the outside of the conical drip part 59. The infusion fluid 3 then flows along this outside to the tip 63 of the inlet valve body 49. Due to the fluid filled cavity 24, the upper slanting part 58 of the wall 54 of the connector 53 and the conical shape of the drip part 59 the infusion fluid leaves the tip 63 shapes as drops 20.

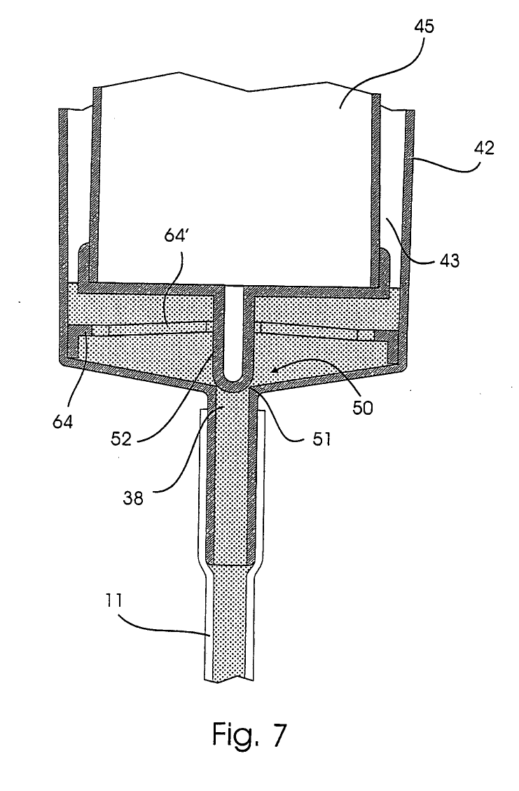

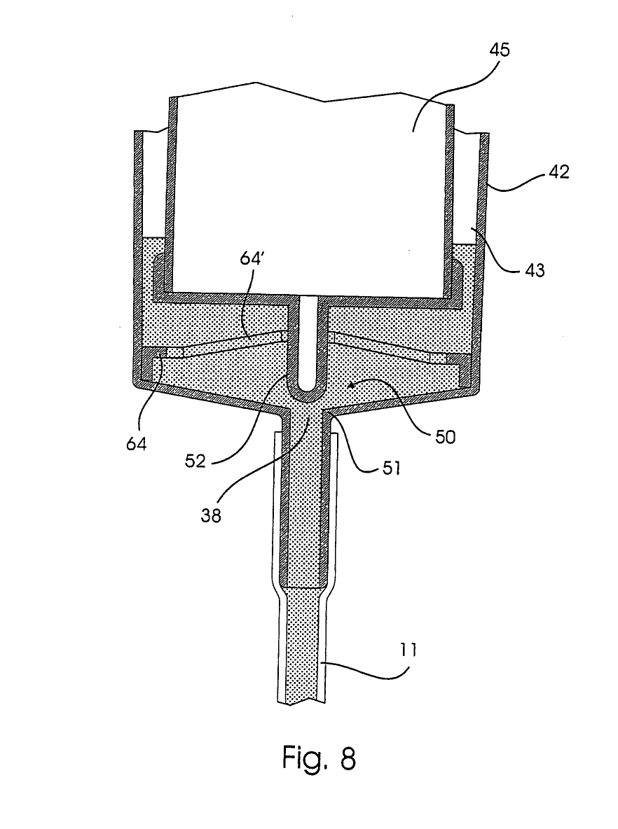

As seen best in FIGS. 7 and 8 a spring 64 functions between the second housing part 42 and the outlet valve body 52 of the outlet valve 50. The spring 64 is formed with a number of through openings 64′.

The spring 64 is prestressed so that the outlet valve body 52 is urged against the outlet valve seat 51 when the level of infusion fluid in the second fluid chamber 43 doesn't exceed a level where the buoyancy of the second floater 45 is the same as the spring forces of the spring 64. The outlet throttle and/or closure valve 50 is then closed as showed in FIG. 7.

The outlet valve body 52 is however lifted away from the outlet valve seat 51 when the level of infusion fluid in the second fluid chamber 43 exceeds the level where the buoyancy of the second floater 45 is the same as the spring forces of the spring 64. This situation is shown in FIG. 8. The outlet throttle and/or closure valve 50 is now open.

The above described arrangement of the outlet throttle and/or closure valve 50 of the housing 42 secures effectively that there always will be a sufficiently column of infusion fluid above the injection needle 4 in the vein of the patient 5 to maintain the pressure needed in e.g. a vain for avoiding back flow of blood into the infusion set 1,33.

Air from the fluid container or the environment can in some cases enter the infusion set or parts of the infusion set when the fluid flow to the patient's body momentarily closes more or less. This can be dangerous if such air passes into e.g. the patients veins together with the fluid. This risk is effectively eliminated as the spring 56 closes the outlet valve 50 before the second fluid chamber is empty.

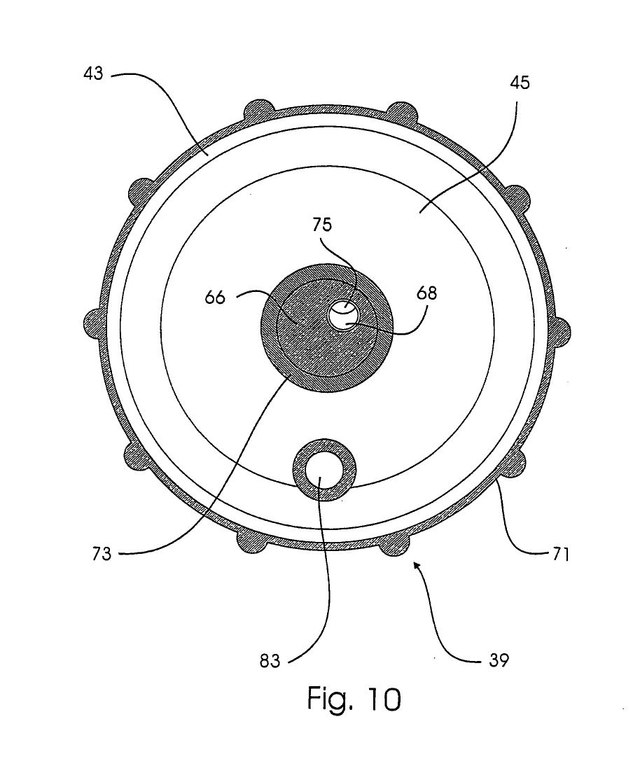

As best seen in FIGS. 9 and 10, a first cup 65 with a first bottom 66 is formed on the lower side 67 of the first housing part 40. A first flow passage 68 is eccentrically formed in the first bottom 66. A protrusion 69 on the lower side 70 of the first floater 45 is extending down into the first cup 65.

The partition 39 comprises a tubular part 71 rotatable connected with the housing parts 40 and 42 and a cross wall 72 extending across the internal side of the tubular part 71. A second cup 73 with a second bottom 74 is formed on the cross wall 72. A second flow passage 75 is eccentrically formed in the second bottom 74.

The above-described construction forms the control valve 46 for allowing fluid communication between the first fluid chamber 41 and the second fluid chamber 43. The control valve 46 is used to regulate the flow of infusion fluid to the patient by simply turning the partition 39 in relation to the housing 35. In FIG. 10 is the control valve only partly open since the two flow passages 68 and 75 only partly is overlapping each other.

A valve body 76, which is co-operating with a valve seat 77, is furthermore formed on the protrusion 69, thereby forming an outlet throttle and/or closure valve 78 at the outlet of the first housing part 40.

A tube 79 is extending downwards against the top 80 of the second floater 45. The object of this tube 79 is to lead the infusion fluid in an even flow over the top of the second floater 45 without disturbing the operation of the infusion set by splashing.

The housing 35 is communicating with the environment via a first ventilation opening 81 formed at the top of the first housing part 40, a second ventilation opening 82 formed in the tubular part 71 of the partition 39 and a third ventilation opening 83 formed in the cross wall 72 of the partition 39.

A ventilation opening (not shown) can alternatively be formed in the tubular part 71 of the partition 39 below the wall 72 instead of the second—and third ventilation openings.

The ventilation openings secure that there always will be the same pressure inside the housing of the flow regulator as outside the housing. The flow resistance in e.g. a vein of the patient does therefore not directly influence the flow of fluid from the fluid container to the flow regulator. A filter (not shown) can optionally be inserted into the ventilation openings of the housing for preventing that the infusion fluid is contaminated by e.g. bacteriological impurities coming from the environment. The flow regulators according to the present invention offer a more accurate and secure infusion procedure end known hitherto.

1. An infusion set for administering an infusion fluid to a patient, comprising
a fluid container for the infusion fluid,
an injection needle for injecting the infusion fluid into the patient's body,
a flow regulator having a housing with an inlet which communicates with the fluid container and an outlet which communicates with the injection needle via a fluid duct,
at least one fluid chamber in the housing, and
at least one floater placed in the at least one fluid chamber,
an inlet throttle that includes a valve seat formed on the housing at the inlet and a corresponding inlet valve body connected with the floater at an axial distance from the at least one floater by at least one connector,
an air gap formed between the inlet valve body and the floater, and
at least one opening in the at least one connector for allowing fluid communication between the inlet throttle and the housing.

2. The infusion set according to claim 1, wherein the flow regulator (6;34) further comprises an outlet throttle that includes an outlet valve seat formed on the housing at the outlet and a corresponding outlet valve body formed on the floater.

3. The infusion set according to claim 1, wherein the flow regulator comprises
a partition, which divides the housing into a first housing part which has an inlet and defines a first fluid chamber, and into a second housing part which has an outlet and is defines a second fluid chamber,
a control valve arranged for allowing fluid communication between the two chambers, and
a first floater placed in the first fluid chamber and a second floater placed in the second fluid chamber.

4. The infusion set according to claim 1, wherein the axial distance between the valve inlet valve body and the floater is larger than the size of a drop of infusion fluid.

5. The infusion set according to claim 1, wherein the inlet valve body has a mainly conical surface with a tip facing the floater and a cavity formed opposite the tip.

6. The infusion set according to claim 5, wherein the cavity is formed in the inlet valve body and fluid connected with the mainly conical outer surface of the inlet valve body via a free edge between the outer surface of the inlet valve body and the cavity.

7. The infusion set according to claim 5, wherein the cavity is formed in the inlet valve body and fluid connected with the mainly conical surface of the inlet valve body via a free edge of a part of the wall of the connector defining an upper wall part, with the edge facing the inlet, and at least one through opening formed in the upper wall part.

8. The infusion set according to claim 1, wherein the at least one connector has a slanting part extending outwards from the mainly conical surface of the inlet valve body into a slanting direction against the inlet of the housing.

9. The infusion set according to claim 1, which further comprises a guide pin extending into the inlet of the housing on the inlet valve body opposite its tip.

10. The infusion set according to claim 1, wherein the flow regulator is arranged so that the at least one floater is allowed a maximal stroke in the housing of the regulator, with the stroke being between 0.1 and 15 mm.

11. The infusion set according to claim 1, which further comprises a ventilation opening at the inlet end of the housing.

12. The infusion set according to claim 1, wherein the flow regulator is biased to urge the outlet throttle against or into its closed position.

13. The infusion set according to claim 12, wherein the biasing is achieved by an elastic means of at least one spring extending between the housing and the second floater.

14. The infusion set according to claim 3, which further comprises a control valve arranged in the partition for allowing fluid communication between the two chambers, with the control valve comprising at least one first flow passage eccentrically formed in the bottom of the first housing part and at least one second flow passage, which when cooperating with the first flow passage is eccentrically provided in the partition, and wherein the partition is rotatably connected with the at least first housing part.

15. The infusion set according to claim 3, wherein the partition comprises a tubular part which is rotatably connected with at least the first housing part, and which further comprises a cross wall extending between the inner side of the tubular part, thereby forming the at least second flow passage in the wall.

16. The infusion set according to claim 3, which further comprises a second ventilation opening in the tubular part of the partition.

17. The infusion set according to claim 1, wherein the at least one of the ventilation openings of the housing is equipped with a filter.
